(19) United States
(12) Patent Application Publication
(10) Pub. No.: US 2007/0218091 A1
(43) Pub. Date: Sep. 20, 2007

(54) Gel with a High Concentration of Branched Sulphonic Polyester and Method For Preparing Same

(75) Inventor: Jonathan Gawtrey, Boulogne (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'OREAL S.A., PARIS (FR)

(21) Appl. No.: 11/587,152

(22) PCT Filed: Apr. 22, 2005
(86) PCT No.: PCT/FR05/01003

§ 371 Date: May 3, 2007

Related U.S. Application Data
(60) Provisional application No. 60/571,916, filed on May 18, 2004.

(30) Foreign Application Priority Data
Apr. 23, 2004 (FR) 0450775

(51) Int. Cl. A61K 8/85; A61K 8/06; A61Q 5/06; C07C 303/28; C07C 309/63

(52) U.S. Cl. 424/401; 424/70.11; 560/14

(57) ABSTRACT

A cosmetic composition in the form of a gel with a high concentration of branched sulphonic polyester and a method for preparing said gel and using same for producing cosmetic compositions in any galenic form.

A subject matter of the invention is a cosmetic composition provided in the form of a gel with a high concentration of branched sulfonic polyester. The invention is also targeted at the process for the manufacture of this gel and at its use in the preparation of cosmetic compositions having any formulation form.

Within the meaning of the present invention, the term “gel” is understood to mean an initially liquid composition which is thickened by virtue of an agent having thickening and/or gelling properties and which has, at the end, a minimum viscosity of 160 cPs, preferably 250 cPs, at 25° C. (Rhéomat 180 viscometer, spindle 2, reading after 30 s) and a maximum viscosity of 10⁶ poises, preferably of 10⁵ poises.

The use of branched sulfonic polyesters in cosmetics is known. In the field of skin care, mention may be made of the patent application filed by the applicant company under the number FR 03/00569, which discloses foaming compositions comprising branched sulfonic polyesters, the concentration of branched polyester by weight advantageously ranging between 20 and 35% by weight, with respect to the total weight of the composition.

In hair cosmetics, mention may be made of the document FR 2 833 491, which relates to the use of branched sulfonic polyesters in aqueous lacquers. Mention may also be made of the documents EP 0 966 946, WO 98/38969 and WO 99/63955, which relate to compositions comprising branched sulfonic polyesters at concentrations by weight ranging from 0.1 to 40%, with respect to the total weight of the composition.

These documents indicate, very generally, that it is possible to employ the sulfonic polyesters in this broad range of concentrations in order to obtain without distinction sprays, foams, gels or lotions. However, the examples give no composition comprising these polyesters at a high concentration and existing in the gel form.

Furthermore, products having care, protection and/or conditioning properties are also sought for the skin, in particular of the human face, and the superficial body growths in particular eyelashes and eyebrows. Such products can be foundations, lipsticks, creams or gels for the body or face, eye shadows, eyeliners, mascaras or blushers.

In addition, whether for the hair or skin, cosmetic compositions capable of forming, on these keratinous substances, a flexible film which follows the movements of the skin or of the hair without an effect of tightness or heaviness or a rigid feeling are also sought.

The problem posed by the present invention is that of providing homogeneous and malleable gels which provide the keratinous substance, in particular the hair, with good cosmetic properties and high fixing; in particular, these gels make it possible to obtain very strong and very long lasting fixing of the hair.

Surprisingly and unexpectedly, the applicant company has found that this problem can be solved by using branched sulfonic polyesters at a high concentration, that is to say of greater than 40% by weight, in an alcoholic or aqueous medium, with respect to the total weight of the composition.

This property is all the more surprising as the preparation of cosmetic compositions based on branched sulfonic polyesters proves to be difficult due to the physical properties of this type of polymer.

Specifically, the preparation of such compositions from a branched sulfonic polyester is difficult to envisage as the dispersion or dissolution of the polyester is lengthy, difficult and problematic in so far as the starting material is available in the form of a block.

In addition, the use of polymer of polyester type at high concentration often results in pastes which are difficult to grasp, the performance of which is most inadequate in terms of cosmetic and fixing properties.

A subject matter of the invention is a gel comprising between 40 and 70% by weight of branched sulfonic polyester, in an aqueous or aqueous/alcoholic medium, with respect to the total weight of the gel.

According to the invention, the expression “between 40 and x % by weight of branched sulfonic polyester” excludes the value 40% by weight of branched sulfonic polyester.

Advantageously, in the aqueous/alcoholic medium, the alcohol is chosen from C₁ to C₄ alcohols and preferably ethanol.

According to the invention, it is essential for the sulfonic polyester to be branched.

Another subject matter of the invention relates to a process for the preparation of a gel comprising between 40 and 70% by weight of branched sulfonic polyester, in an aqueous or aqueous/alcoholic medium, with respect to the total weight of the composition, characterized in that it comprises a stage of heating, between 45° C. and 120° C., a composition comprising the branched sulfonic polyester in the aqueous medium.

According to one embodiment of the process according to the invention, a C₁ to C₄ alcohol, in particular ethanol, is added to said composition, optionally after stirring and cooling, so that the concentration of C₁ to C₄ alcohol, in particular of ethanol, in the gel is between 0 and 20% by weight.

Generally, the branched sulfonic polyester employed as starting material in the process according to the invention is generally provided in the form of crushed solid block(s).

Advantageously, the process according to the invention comprises heating, between 45° C. and 120° C., a composition comprising, as relative percentage by weight:
between 40 and 70% of branched sulfonic polyester,
between 30 and 60% of water,
   optionally, after stirring and cooling, a C₁ to C₄ alcohol, in particular ethanol, is added so that the concentration of C₁ to C₄ alcohol, in particular of ethanol, in the gel is between 0 and 20% by weight.

Yet another subject matter of the invention relates to a process for the preparation of a cosmetic composition which is provided in any formulation form, characterized in that it comprises the use and the heating of an aqueous or aqueous/alcoholic gel comprising between 40 and 70% by weight of branched sulfonic polyester, with respect to the total weight of the gel.

Yet another subject matter of the invention relates to the use of the gel as product for the fixing the and/or form retention of the hair or for the manufacture of such products.

Advantageously, the glass transition temperature of the sulfonic polyesters is between −15 and +10° C., preferably between −12 and +7° C., more advantageously between −10 and +5° C. Advantageously, the gel comprises, in a concentration by weight with respect to the total weight of the composition, between 40 and 65% by weight of branched sulfonic polyester, more advantageously between 40 and 60% and more advantageously still between 45 and 58%.

Preferably, the branched sulfonic polyester is a polymer formed by the polycondensation

a) of at least one dicarboxylic acid not carrying a sulfonic functional group,

b) of at least one diol or of a mixture of a diol and of an amine,

c) of at least one monomer comprising two reactive functional groups, which are identical or different, chosen from hydroxyl, amino and carboxyl groups and additionally carrying at least one sulfonic functional group, and

d) of at least one monomer comprising at least three reactive functional groups, which are identical or different, chosen from hydroxyl, amino and carboxyl groups.

In the process for the preparation of the branched sulfonic polyester, the monomer(s) d) bring about the branching of the sulfonic polyester.

More preferably, the branched sulfonic polyester additionally comprises units derived from difunctional monomers (e) chosen from hydroxycarboxylic acids and aminocarboxylic acids or a mixture of these.

Preferably, the difunctional monomer (e) represents up to 40 mol % of the combined monomers; the monomer (c) carrying at least one sulfonic functional group represents from 2 to 15 mol % of the combined monomers; the monomer (d) comprising at least three reactive functional groups represents from 0.1 to 40 mol %, with respect to the combined monomers.

Advantageously, the dicarboxylic acids not carrying a sulfonic functional group forming the units (a) are chosen from aliphatic dicarboxylic acids, alicyclic dicarboxylic acids, aromatic dicarboxylic acids and mixtures of such acids.

More advantageously, the dicarboxylic acids forming the units (a) are chosen from the group formed by 1,4-cyclohexanedioic acid, succinic acid, glutaric acid, adipic acid, azelaic acid, sebacic acid, fumaric acid, maleic acid, 1,3-cyclohexanedioic acid, phthalic acid, terephthalic acid and isophthalic acid, and a mixture of such acids.

Advantageously, the diols forming the units (b) are chosen from alkanediols and polyalkylenediols and are chosen from ethylene glycol, propylene glycol, diethylene glycol, triethylene glycol and polypropylene glycol.

In particular, the diamines forming the units (b) are chosen from alkanediamines and poly(oxyalkylene)diamines.

Advantageously, the monomer (c) carrying at least one sulfonic functional group is chosen from dicarboxylic acids, esters of dicarboxylic acids, glycols and hydroxy acids all carrying at least one sulfonic group in the acid and/or neutralized form, preferably in the neutralized form.

Advantageously, the polyfunctional monomer (d) is chosen from trimethylolethane, trimethylolpropane, glycerol, pentaerythritol, sorbitol, trimellitic anhydride, erythritol, threitol, dipentaerythritol, pyromellitic dianhydride and dimethylpropionic acid.

Advantageously, in the process for the preparation of a gel according to the invention, the temperature is between 55° C. and 100° C., preferably between 70° C. and 90° C. Preferably, the heating is carried out with stirring.

Advantageously, in the process for the preparation of a cosmetic composition provided in any formulation form, the temperature is between 55° C. and 100° C., more preferably between 70° C. and 90° C. Preferably, the heating is carried out with stirring and the aqueous or aqueous/alcoholic gel is employed in dispersion in a cosmetically acceptable medium.

The term “sulfonic functional group” of the units (c) encompasses both the sulfonic acid functional group (—SO₃H) and the corresponding salified functional groups obtained by neutralization of the sulfonic acid functional group with a base, for example with an alkali metal hydroxide.

The branched sulfonic polyesters can comprise, in addition to the four types of units (a) to (d) described above, units (e) derived from monomers comprising two different reactive functional groups chosen, for example, from hydroxycarboxylic acids and aminocarboxylic acids or a mixture of these.

These units (e) can represent up to 40 mol % of the combined monomers (a), (b), (c), (d) and (e).

Of course, the branched sulfonic polymers used in the present invention are preferably obtained from a mixture of monomers in which the number of equivalents of carboxylic acid functional groups is substantially equal to the number of equivalents of hydroxyl functional groups and of amino functional groups optionally present.

The sulfonic polymers used in the process of the invention are known and are commercially available, for example from Eastman. Among these polymers, preference will be given to those sold under the names AQ 1045, AQ 1350 and AQ 14000. Mention may be made, as particularly preferred commercial product, of the product sold under the name AQ 1350 by Eastman. The process for the preparation of the sulfonic polyester employed according to the invention is, for example, disclosed in U.S. Pat. No. 5,543,488. The cosmetic compositions prepared according to the process of the present invention can additionally comprise one or more additional solvents.

These solvents may or may not be volatile and they encompass in particular water, C₂₋₁₈ alcohols which may be aliphatic or aromatic, C₂₋₃₀ polyols, C₄₋₁₈ alkanes, volatile or nonvolatile, fluid, linear or cyclic silicones, acetone, methyl ethyl ketone, methyl acetate, butyl acetate, ethyl acetate, dimethoxyethane or diethoxyethane.

The additional solvents which have proved to be particularly advantageous for use in the cosmetic compositions of the present invention are volatile silicones, in particular volatile cyclic silicones, such as decamethylpentasiloxane (D₅).

The cosmetic compositions prepared according to the process of the present invention can additionally comprise one or more cosmetic active principles and/or one or more formulation additives. Mention may be made, as such active principles and additives, of mineral, vegetable or animal oils, waxes, which may or may not be oxyethylenated, paraffins, fatty acids, fatty amides, fatty esters, fatty alcohols, reducing agents, oxidizing agents, sequestering agents, thickening agents, softening agents, antifoaming agents, moisturizing agents, emollients, agents for adjusting the pH, plasticizers, sunscreens, direct dyes, oxidation dye precursors (bases and couplers), pigments, inorganic fillers, clays, pearlescent agents, fragrances, peptizing agents, preservatives, anionic, cationic, nonionic, amphoteric and zwitterionic surface-active agents, fixing polymers, conditioning polymers, proteins or vitamins.

A person skilled in the art will take care to choose the additional solvent or solvents, the active principle or principles and/or the formulation additive or additives so as not to harm the intrinsic advantageous properties of the compositions of the present invention.

The compositions prepared according to the process of the present invention can be provided in any form which makes possible ease of application to and uniform distribution over the hair. The composition can, for example, be a lotion, a foam, a gel or an emulsion. These compositions can be applied according to a rinse-out form or a leave-in form, preferably a leave-in form.

The process for the preparation of a composition provided in any formulation form comprises the preparation of the aqueous or aqueous/alcoholic gel by heating, between 45° C. and 120° C., a composition comprising, as relative percentage by weight:
between 40 and 70% of branched sulfonic polyester,
between 30 and 60% of water,
   optionally, after stirring and cooling, a C₁ to C₄ alcohol, in particular ethanol, is added so that the concentration of C₁ to C₄ alcohol, in particular of ethanol, in the gel is between 0 and 20% by weight.

It is possible to envisage the packaging of these compositions in a device which makes possible application by spraying, such as a pump-action spray or an aerosol device in the presence of at least one propellant. This propellant is preferably chosen from air, carbon dioxide, nitrogen, dimethyl ether, halogenated hydrocarbons and mixtures of these.

Another subject matter of the present invention relates to a cosmetic process for caring for and/or protecting and/or making up the skin, including the scalp, and/or the keratinous fibers of human beings, such as the hair, eyelashes and eyebrows, comprising the application of a cosmetic composition in the gel form as defined above to the skin and/or said keratinous fibers of human beings. The care of the skin can consist in particular of the repair or the prevention of the skin against the damaging effects of time, such as dryness, wrinkles and fine lines, or flabbiness. The protection of the skin is aimed in particular at the protection of the latter against external stimuli, such as the wind, pollution or UV radiation.

Another subject matter of the present invention relates to a cosmetic process for conditioning human keratinous substances and in particular the skin, including the scalp, and/or human keratinous fibers, comprising the application of a cosmetic composition as defined above to these keratinous substances.

Yet another subject matter of the present invention relates to the use of this composition in the gel form for the manufacture of cosmetic compositions intended for caring for and/or protecting and/or making up human keratinous substances, such as the skin, hair, eyelashes, nails or lips. More especially, a further subject matter of the invention is the use of this composition for the manufacture of a hair cosmetic product for the purpose of retaining the form of and/or fixing the hairstyle.

The concentration of branched sulfonic polyester in the cosmetic products prepared from the starting gel is between 0.1 and 39%, preferably between 0.5 and 25%, with respect to the total weight of the cosmetic composition.

Another subject matter of the invention relates to the use of the gel for caring for and/or protecting and/or making up human keratinous substances, such as the skin, hair, eyelashes, nails or lips.

This gel can also be used in the field of the following industries: paper, construction, adhesives, copiers, household appliances, detergents, wood, metallurgy, paint, coating, plastics, leather, textile, motor vehicle, aeronautics, electronics, computing, disposable paper products, such as diapers, packaging, oil or dressings.

The examples below illustrate the present invention without limiting the scope thereof.

In the examples, AM means active material and the percentages are expressed by weight with respect to the total weight of the composition.

### EXAMPLES

1. Preparation of a Gel Concentrated in Branched Sulfonic Polyester

| | |
|---|---|
| Branched sulfonic polyester, | 43.5% AM |
| AQ 1350 ® (Eastman) | |
| Water | q.s. for 100% |

The process for preparing the aqueous gel of a sulfonic polyester consists in dispersing the AQ 1350 in the water under hot conditions (80° C.).

This preparation of the branched sulfonic polyester facilitates the industrial use of AQ 1350. The resulting gel can be used in the preparation of aqueous cosmetic compositions based on AQ 1350.

2. Aqueous/Alcoholic gel of AQ 1350

| | |
|---|---|
| Branched sulfonic polyester, | 47.5% AM |
| AQ 1350 (Eastman) | |
| Ethanol | 15% |
| Water | q.s. for 100% |

The process for producing the aqueous/alcoholic gel consists in dispersing the AQ 1350 in water with stirring under hot conditions (80° C.). The combined product is subsequently cooled and the ethanol is added with stirring.

What is claimed is:

1. A gel comprising between 40 and 70% by weight of branched sulfonic polyester, in an aqueous or aqueous/alcoholic medium, with respect to the total weight of the gel.

2. The gel as claimed in claim 1, characterized in that the glass transition temperature of the sulfonic polyesters is between −15 and +10° C.

3. The gel as claimed in claim 1 or 2, characterized in that the branched sulfonic polyester is a polymer formed by the polycondensation
a) of at least one dicarboxylic acid not carrying a sulfonic functional group,
b) of at least one diol or of a mixture of a diol and of an amine,
c) of at least one monomer comprising two reactive functional groups, which are identical or different, chosen from hydroxyl, amino and carboxyl groups and additionally carrying at least one sulfonic functional group, and
d) of at least one monomer comprising at least three reactive functional groups, which are identical or different, chosen from hydroxyl, amino and carboxyl groups.

4. The gel as claimed in any one of the preceding claims, characterized in that the branched sulfonic polyester additionally comprises units derived from difunctional monomers (e) chosen from hydroxycarboxylic acids and aminocarboxylic acids or a mixture of these.

5. The gel as claimed in claim 4, characterized in that the difunctional monomer (e) represents up to 40 mol % of the combined monomers.

6. The gel as claimed in any one of claims 3 to 5, characterized in that the monomer (c) carrying at least one sulfonic functional group represents from 2 to 15 mol % of the combined monomers.

7. The gel as claimed in any one of claims 3 to 6, characterized in that the monomer (d) comprising at least three reactive functional groups represents from 0.1 to 40 mol % with respect to the combined monomers.

8. The gel as claimed in any one of claims 3 to 7, characterized in that the dicarboxylic acids not carrying a sulfonic functional group forming the units (a) are chosen from aliphatic dicarboxylic acids, alicyclic dicarboxylic acids, aromatic dicarboxylic acids and mixtures of such acids.

9. The gel as claimed in any one of claims 3 to 8, characterized in that the dicarboxylic acids forming the units (a) are chosen from the group formed by 1,4-cyclohexanedioic acid, succinic acid, glutaric acid, adipic acid, azelaic acid, sebacic acid, fumaric acid, maleic acid, 1,3-cyclohexanedioic acid, phthalic acid, terephthalic acid and isophthalic acid, and a mixture of such acids.

10. The gel as claimed in any one of claims 3 to 9, characterized in that the diols forming the units (b) are chosen from alkanediols and polyalkylenediols.

11. The gel as claimed in any one of claims 3 to 10, characterized in that the diols forming the units (b) are chosen from ethylene glycol, propylene glycol, diethylene glycol, triethylene glycol and polypropylene glycol.

12. The gel as claimed in any one of claims 3 to 11, characterized in that the diamines forming the units (b) are chosen from alkanediamines and poly(oxyalkylene)diamines.

13. The gel as claimed in any one of claims 3 to 12, characterized in that the monomer (c) carrying at least one sulfonic functional group is chosen from dicarboxylic acids, esters of dicarboxylic acids, glycols and hydroxy acids all carrying at least one sulfonic group in the acid and/or neutralized form, preferably in the neutralized form.

14. The gel as claimed in any one of claims 3 to 13, characterized in that the polyfunctional monomer (d) is chosen from trimethylolethane, trimethylol-propane, glycerol, pentaerythritol, sorbitol, trimellitic anhydride, erythritol, threitol, dipentaerythritol, pyromellitic dianhydride and dimethylpropionic acid.

15. The gel as claimed in any one of the preceding claims, characterized in that it comprises, in a concentration by weight with respect to the total weight of the composition, between 40 and 65% by weight of branched sulfonic polyester, more advantageously between 40 and 60% and more advantageously still between 45 and 58%.

16. A process for the preparation of a gel as claimed in any one of the preceding claims, characterized in that it comprises a stage of heating, between 45° C. and 120° C., a composition comprising the branched sulfonic polyester in the aqueous medium.

17. The process as claimed in claim 16, characterized in that a C₁ to C₄ alcohol, in particular ethanol, is added to said composition, optionally after stirring and cooling, so that the concentration of C₁ to C₄ alcohol, in particular of ethanol, in the gel is between 0 and 20% by weight.

18. The process as claimed in either one of claims 16 and 17, characterized in that it comprises heating, between 45° C. and 120° C., a composition comprising, as relative percentage by weight:
between 40 and 70% of branched sulfonic polyester,
between 30 and 60% of water,
optionally, after stirring and cooling, a C₁ to C₄ alcohol, in particular ethanol, is added so that the concentration of C₁ to C₄ alcohol, in particular of ethanol, in the gel is between 0 and 20% by weight.

19. The process as claimed in any one of claims 16 to 18, characterized in that the temperature is between 55° C. and 100° C.

20. The process as claimed in any one of claims 16 to 19, characterized in that the temperature is between 70° C. and 90° C.

21. The process as claimed in any one of claims 16 to 20, characterized in that the heating is carried out with stirring.

22. The process as claimed in any one of claims 16 to 21, characterized in that the glass transition temperature of the sulfonic polyesters is between −15 and +10° C.

23. A process for the preparation of a cosmetic composition which is provided in any formulation form, characterized in that it comprises the use and the heating of an aqueous or aqueous/alcoholic gel comprising between 40 and 70% by weight of branched sulfonic polyester, with respect to the total weight of the gel.

24. The process as claimed in claim 23, characterized in that it comprises the preparation of the aqueous or aqueous/alcoholic gel by heating, between 45° C. and 120° C., a composition comprising, as relative percentage by weight:
between 40 and 70% of branched sulfonic polyester,
between 30 and 60% of water,
optionally, after stirring and cooling, a C₁ to C₄ alcohol, in particular ethanol, is added so that the concentration of C₁ to C₄ alcohol, in particular of ethanol, in the gel is between 0 and 20% by weight.

25. The process as claimed in either one of claims 23 and 24, characterized in that the temperature is between 55° C. and 100° C.

26. The process as claimed in any one of claims 23 to 25, characterized in that the temperature is between 70° C. and 90° C.

27. The process as claimed in any one of claims 23 to 26, characterized in that the heating is carried out with stirring.

28. The process as claimed in any one of claims 23 to 27, characterized in that the aqueous or aqueous/alcoholic gel is employed in dispersion in a cosmetically acceptable medium.

29. The use of a gel as claimed in any one of claims 1 to 15 of the gel as product for the fixing and/or form retention of the hair or for the manufacture of such product.

30. The use of a gel as claimed in any one of claims 1 to 15 for the manufacture of cosmetic compositions intended for caring for and/or protecting and/or making up human keratinous substances, such as the skin, hair, eyelashes, nails or lips.

31. The use of the gel as claimed in any one of claims 1 to 15 for caring for and/or protecting and/or making up human keratinous substances, such as the skin, hair, eyelashes, nails or lips.
